(19) United States
(12) Patent Application Publication
(10) Pub. No.: US 2025/0381058 A1
(43) Pub. Date: Dec. 18, 2025

(54) A CATHETER BAG AND CONNECTIONS AND ACCESSORIES THEREFOR

(71) Applicant: CLINISUPPLIES LIMITED, Watford (GB)

(72) Inventors: Martin Richard Beynon, Watford (GB); Georgina Elizabeth Bird, Watford (GB)

(21) Appl. No.: 18/879,492

(22) PCT Filed: Jul. 5, 2023
(86) PCT No.: PCT/GB2023/051765

§ 371 Date: Dec. 27, 2024

(30) Foreign Application Priority Data
Jul. 5, 2022 (GB) 2209851.1

(51) Int. Cl. A61F 5/451; A61F 5/44

(52) U.S. Cl. CPC A61F 5/451; A61F 5/4404

(57) ABSTRACT

A system comprising a urine drainage bag comprising overlapping bag walls, sealed together about their periphery, defining an internal collection volume and having an inlet, an inlet tube entering via the inlet and sealingly secured within the urine drainage bag having a first end opening into the collection volume, and a second end operably connected, in use, to a user via a catheter, sheath or the like; the urine drainage bag having a second outlet to allow urine to be drained from the urine drainage bag, said second outlet being releasably closable.

### FIELD OF THE INVENTION

The present invention relates to a urine drainage bag and accessories associated therewith. In particular, the invention is concerned with an improved urine drainage bag along with accessories such as connectors and closure means for use with said bag.

### BACKGROUND TO THE INVENTION

The use of urinary catheters worldwide is considerable and has been estimated to be around 100 million. Typically, a small tube is inserted via the urethra into the bladder to facilitate emptying of the bladder where this is difficult for a person either to carry out or to control, although their use to deliver medicines directly into the bladder is also common. In the former use, the catheter tube is usually fluidly attached to a replaceable urine drainage bag which collects the urine. The bag ensures there is an appropriate receptacle to capture the free flowing urine, allowing the catheter to remain in place within the patient, with only the bag needing to be emptied and/or replaced. To reduce any leakage during the changing process, a valve is included in the system to close off tubing whilst a bag is temporarily not present. However, many prior catheter valves are difficult to operate especially by a person having poor dexterity.

Where a person has urinary incontinence then a catheter and attached bag are permanently in place. With such long term use, there is not only the problem of an increased risk of infection, but also the problem of inconvenience to the person both in terms of having to have a bag about them at all times and hygiene problems. Particularly when changing a bag, there is a risk of a small amount of urine escaping the bag and tubing causing distress and also—leakage onto clothing or furniture. There is also the opportunity for bacteria to enter the catheter when changing a bag.

Although procedures are usually in place for dealing with the changing and emptying of bags, the risk of urinary tract infections remains high.

In addition to the health and hygiene aspects outlined above, there is also an aesthetic aspect which is often neglected when a person is being fitted with a catheter tube and bag. Bags commonly in use are designed from a purely functional aspect and any attempt at minimising the clinical look to a urine drainage bag, in particular, is ignored. For example, the bag is either transparent, meaning the urine therein is visible or a bright white colour which can show through many fabrics. Many of those who permanently have a catheter fitted are therefore deterred from going out in public out of a feeling of embarrassment.

It is an object of the current invention to provide a urine drainage bag and system which addresses the above problems and also enables simpler and more sanitary operation.

### SUMMARY OF THE INVENTION

According to a first independent aspect of the invention there is a system comprising a urine drainage bag comprising overlapping bag walls, sealed together about their periphery, defining an internal collection volume and having an inlet,
an inlet tube entering via the inlet and sealingly secured within the urine drainage bag having a first end opening into the collection volume, and a second end operably connected, in use, to a user via a catheter, sheath or the like;
the urine drainage bag having a second outlet to allow urine to be drained from the urine drainage bag, said second outlet being releasably closable.

The second outlet optionally comprises a spout closable by a flip cap, secured to the drainage bag, conveniently by means of a living hinge. The flip cap further optionally includes a plug, of complementary shape to the inside of the spout to achieve a push-fit connection with the spout. The plug yet further optionally comprises one or more circumferential rings to aid engagement with the spout, whereby in the case of two or more ring, said rings are concentric.

The flip cap optionally has a tab to aid manipulation by a user. The tab further optionally defines a throughaperture, the outer surface of the spout including a nodule to engage the walls of the throughaperture and releasably retain the tab against the spout.

Optionally, the second outlet comprises a spout closable by a removable insertable stopper, the insertable stopper further optionally including a ring to aid the user in removing the stopper. Yet further optionally, the insertable stopper has a hollow shaft to allow for the plug to be machine insertable such that once it has been removed it cannot be reinserted by hand.

Preferably the second outlet comprises a drainage tube sealingly secured within the urine drainage bag having a first end of the drainage tube opening into the collection volume and a tap assembly on the second end of the drainage tube to control release of urine from the second outlet. Said tap assembly further preferably comprises a tap body formed as first and second cylindrical bodies, mutually intersecting at right-angles, the wall of the first cylindrical body defining mutually opposed apertures, aligned with the axis of the second cylindrical body, a tap member rotatably housed in the first cylindrical body movable between a first position preventing fluid flow along the second cylindrical body and a second position allowing fluid flow.

Preferably, a first connector extends from a first end of the second cylindrical body, said first connector further preferably having a plurality of nested frusto-conical sections to enable the tap assembly to be sealingly and securely removably inserted into a tube. Alternatively preferably, the first connector has a single frusto-conical section.

Preferably a re-closable closure extends from a second end of the second cylindrical body to provide additional protection against unwanted leakage from the drainage bag.

Preferably the system includes a clip operably connected to the second end of either the inlet tube or the drainage tube, to allow the length of the inlet tube or drainage tube to be extended, the clip comprising a central cylindrical body defining a lumen through which fluid can flow, the clip having at a first end a flange supporting a sealing element, to for example engage the end of a tube to be inserted into the body, the clip further having one or more securing arms resiliently mounted movable between a first gripping position and a second release position, the arm being biased to the gripping position and released therefrom by pressure applied to a first end of the arm.

A central portion of the bag walls are preferably sealed together, said sealed central region comprising one or more elongate slits which in use can receive a tap assembly or tube to conveniently stow a tap assembly or tube.

Preferably, the bag walls are sealed together in one or more edge regions forming one or more handles. Further preferably a handle comprises one or more elongate slits which allow a handle to be curved more easily around a user's leg.

Preferably, the drainage bag is housed within a sleeve to enable the weight of the drainage bag to be better distributed.

Optionally the drainage bag is formed of a polyvinylchloride material for durability.

The surface of the drainage bag is preferably patterned to reduce the visibility of the drainage bag.

According to a second independent aspect of the invention, there is provided a tap assembly for a system comprising a urine drainage bag defining a urine collection volume, the system comprising a drainage tube having a first end of the drainage tube for entering into the collection volume, wherein the tap assembly is securable on the second end of the drainage tube to control release of urine from the second outlet, wherein, in use, the tap assembly connects the system to a further drainage element of the system allowing urine to flow when the tap is secured on the second end, and when the tap assembly is disconnected, urine flow is prevented. In other words, the tap assembly “locks” the system allowing urine to flow when is secured in place; when the tap assembly is unlocked or disconnected from the system, the urine flow will be stopped. This is advantageous over known ‘push-fit’ elements for example.

Preferably, the first connector extends from a first end of the second cylindrical body.

In some embodiments, the first connector comprises a plurality of nested frusto-conical sections. In alternative embodiments, the first connector has a single frusto-conical section.

Preferably, a re-closable closure extends from a second end of the second cylindrical body.

According to a third independent aspect of the invention, there is provided a tap assembly for a urine drainage system, the tap assembly comprising a tap body formed as first and second cylindrical bodies, mutually intersecting at right-angles, the wall of the first cylindrical body defining mutually opposed apertures, aligned with the axis of the second cylindrical body, a tap member rotatably housed in the first cylinder movable between a first position preventing fluid flow along the second cylindrical body and a second position allowing fluid flow, the tap member actuable by a tap lever.

Preferably the tap lever has a curved surface.

Further preferably, the tap lever comprises a notch. The notch enables the functionality of securing the tap assembly in use, by ‘clicking’ it in place, at the same time that the tap assembly locks the system to prevent the flow of urine.

Preferably, the tap lever comprises a protrusion on the underside of the tap lever. Further preferably, the second cylinder of the tap assembly comprises a complementary recess to the protrusion situated on the tap lever. The protrusion, when situated within the recess, enables the functionality of locking the tap lever in use to prevent leakage of urine if the tap assembly is left partially open. Further preferably the action of slotting the protrusion into the complementary recess will provide an audible click. This will notify the user that the tap lever is now locked in position. Further preferably the act of slotting the protrusion over the rim and into the complementary recess provides a physical indicator that the tap lever is in the locked position. Advantageously, this provides security to those who are hard of hearing to know that the tap lever is in the locked position.

In a further aspect, there is provided a urine drainage system comprising a tap assembly as defined above.

In a further aspect, there is provided a modular urine drainage system comprising a system or tap assembly as defined above.

In a further aspect, there is provided a kit of parts comprising a system and a tap assembly as defined above.

It will be appreciated that each of the dependent aspects are applicable to any of the independent aspects.

### BRIEF DESCRIPTION OF THE DRAWINGS

The invention is now described with respect to the accompanying drawings, which show by way of example only embodiments of a bag and connectors for use therewith. In the drawings:

FIG. 1 illustrates a first embodiment of a urine drainage bag;

FIG. 2 illustrates a second embodiment of a urine drainage bag suitable for use as a night bag;

FIG. 3 is a sectional view through the night bag of FIG. 2 with the bag flip cap in the open position;

FIG. 4 is a sectional view through the night bag of FIG. 2 with a second embodiment of single use bag plug;

FIG. 5 illustrates a second embodiment of leg bag assembly;

FIGS. 6a-c show the second embodiment of leg bag of FIG. 5 in further detail, with 6b and 6c showing more detail of the material in FIG. 6a;

FIG. 7 illustrates the urine drainage bag of FIG. 5 having a shorter connection to the user;

FIG. 8 is a perspective view of a first embodiment of catheter valve assembly;

FIG. 9 is an exploded view of the catheter valve assembly of FIG. 8;

FIG. 10 is a perspective view of a second embodiment of lever tap assembly;

FIG. 11 is an exploded view of the lever tap assembly of FIG. 10;

FIGS. 12a-f are respectively a perspective, first side, second side, third side, end and sectional view through B-B of 12e of a tap body;

FIGS. 13a-f are respectively a perspective, first side, second side, end, bottom and sectional view through A-A of 13e of a tap lever;

FIGS. 14a-d are respectively a perspective, first end, side, second end, and sectional view through A-A of 14c of a tap body tube insert as illustrated in FIGS. 10 and 11;

FIGS. 15a-e are respectively a perspective, side, end, and sectional view through A-A of 15c of a second embodiment of a tube connector insert as illustrated in FIG. 3;

FIGS. 16a-e are respectively a perspective, first end, side, second end, and sectional view through A-A of 16d of a tap body tube insert;

FIGS. 17a-e are respectively a perspective, first end, side, second end, and sectional view through A-A of 17d of a catheter connector insert;

FIGS. 18a-e are respectively a perspective, side, top, section through A-A of 18c and end view of a tap cap;

FIGS. 19a-d are respectively a perspective, side, top, and end view of a first embodiment of bag flip cap;

FIGS. 20a-d are respectively a perspective, side, top, and end view of a second embodiment of bag flip cap

FIGS. 21a-e are respectively a perspective, side, bottom, side and sectional view through D-D of 21d and end view of a further embodiment of a bag stopper;

FIGS. 22a-e are respectively a perspective, top view, first side, second side, end and bottom view of a first embodiment of connection clip;

FIG. 23 illustrates an extension tube assembly;

FIGS. 24a-e are respectively a top view, first perspective, second perspective, first side, bottom view and second side, of a second embodiment of connection clip;

FIGS. 25a and 25b are, respectively, a perspective and end view of a first embodiment of a non-blocking tube; and

FIGS. 26a and 26b are respectively, a perspective and end view of a second embodiment of a non-blocking tube.

FIGS. 27a-c are, respectively, a perspective view from the top and a side view of a third embodiment of a catheter valve assembly and a perspective view of a second embodiment of a tap lever.

FIGS. 28a and 27b are respectively a first side and second side, of a third embodiment of a catheter valve assembly.

FIGS. 29a and 29b are respectively, a side view and a top view a second embodiment of a tap lever.

### DETAILED DESCRIPTION OF THE INVENTION

Conventional systems for collecting urine draining from a person's bladder via a catheter tube inserted into the bladder suffer from a number of disadvantages. First, the various connectors currently used are often difficult to handle and secure together, especially where a person has limited strength and hand/eye co-ordination. Second, many bags have a drainage tap and tube, which can drag on the floor, release a small amount of urine held by surface tension in the tube, and also cut into a patient's often fragile skin. Third many connectors have a syringe port which is rarely used and simply adds to the bulk. Fourth, many bags are designed to be functional, with no concern for any aesthetic considerations. The present invention seeks to provide a urine drainage bag and connection system which is both more practical to employ and wear, more easily and hygienically connected and released from its in-use position and more aesthetically pleasing.

Generally, two types of urine drainage bag are employed. The first type, often referred to as a leg bag is intended to be worn during the day, allowing a person to move around, typically with the bag strapped to their leg. This allows a person to remain mobile and lead a normal life. The leg bag can be worn under clothes, secured to the thigh, knee or lower leg, and is not usually visible to others. The bag is usually secured to the user by elastic straps. Care should be taken on fitting the straps as too loose a fit will allow the bag to slip, whereas too tight a fit can cause chafing and soreness and reduce blood flow. The thickness of the strap is also important as too thin a strap can cut into the user's skin. A leg bag can be encased in a sleeve which distributes the weight of the bag more evenly. A leg bag often includes a tap to allow urine to be drained therefrom, so the leg bag can continue to be used.

The second type is a night bag, which is typically of greater capacity than the leg bag, and used during the night. Often the night bag is designed to be secured to a stand which is located next to the bed in which the user is sleeping. Once drainage has been completed, the tap is closed so the bag can continue collecting. Another difference is the size of a bag: a leg bag usually has a fluid capacity of 300 ml-1 l, whereas a night bag has a capacity of 2 l-4 l. It is also known for the night bag to be used in series with a leg bag.

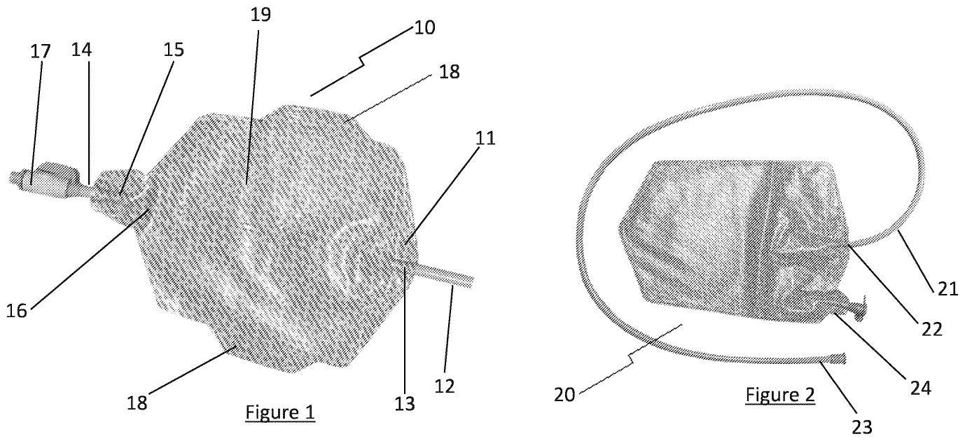

Referring now to FIG. 1, this illustrates a first embodiment of a leg bag, generally referenced 10. The leg bag 10 is designed to be worn during the day, usually secured to the user by means of one or more straps, allowing the user to move from place to place. A leg bag is usually, used for 5-7 days, disposed of and replaced by a new leg bag. The leg bag 10 is typically formed of a plastics material such as polyvinylchloride (PVC). At a first end 11 of the leg bag 10 is a first tube 12, which is sealingly secured to the leg bag 10, with the first end 13 of the first tube 12 inside the leg bag 10, being fluidly connected to the inside of the leg bag 10. The second end of the first tube 12 is operably connected to a catheter or sheath which acts to drain fluid from the bladder of the person using the leg bag 10. A second tube 14 is sealingly secured to a second end 15 of the leg bag 10, with the first end 16 of the second tube 14 inside the leg bag 10. A tap assembly 17 is housed on the second end of the second tube 14, the opening of which tap assembly 17 allows fluid collected in the leg bag 10 to be drained therefrom. Handles 18 allow the leg bag 10 to be secured to the leg with a leg bag strap.

The surface of the leg bag 10, or the leg bag 10 itself is patterned which acts to mask the contents from view and also to break up the outline of the bag so rendering the bag and its contents less visible through clothing.

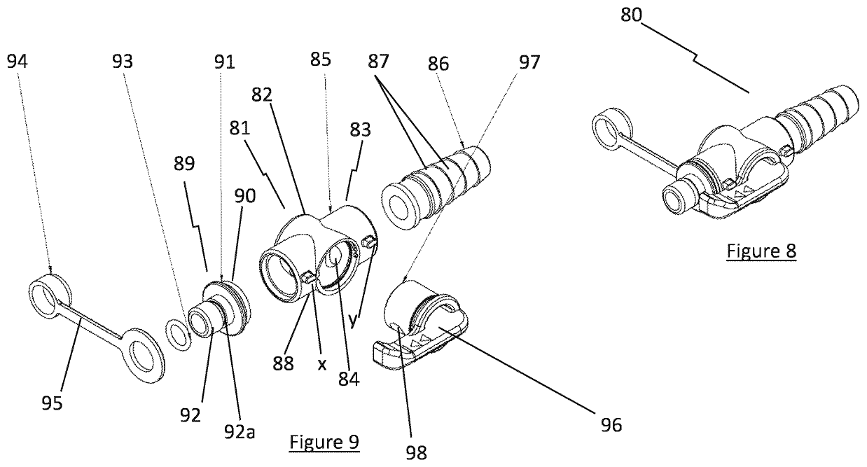

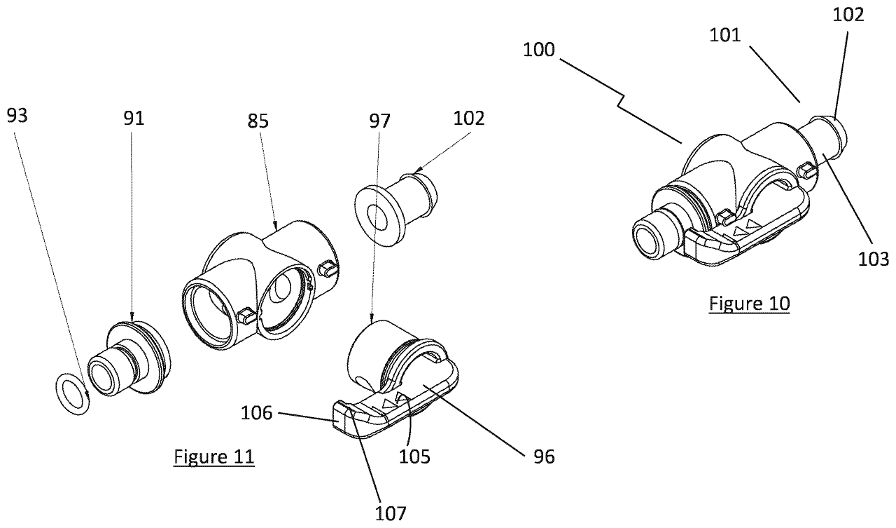

In FIG. 2 is shown a first embodiment of a night bag, generally referenced 20. A night bag 20 typically has a higher capacity than a leg bag and is intended primarily for use during the night or also for those who are bed-bound as it allows for a longer period before the night bag 20 needs to be emptied. Moreover, a night bag is generally used again once it has been emptied, typically being used in total for 5-7 days. Similarly to the leg bag 10, the night bag 20 has a tube 21 sealingly secured into the night bag 20 at a first end 22, with the second end 23 of the tube 21 being operably secured to the catheter valve or leg bag, by means of the lever tap or valve as shown in FIGS. 8 and 10. The night bag 20 also includes a spout 24 which allows the night bag 20 to be emptied. A night bag can either be used by itself to collect urine, or connected to the outlet of a leg bag to provide additional volume for collection.

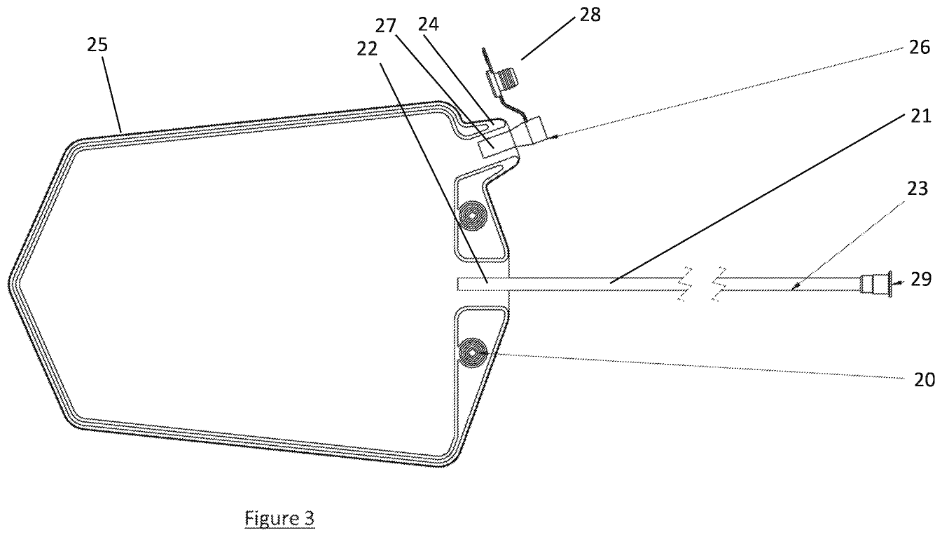

The night bag 20 is shown in FIG. 3 in sectional view. The wall of the night bag 20 is flexible to allow the wall 25 of the night bag 20 to move as fluid flows into the night bag 20. The first end 22 of the tube 21 is bonded in a fluid-tight manner to the wall 25. This prevents fluid and odours from exiting the night bag 20. The wall 25 is formed into a spout 24 which provides egress for fluid from the night bag 20 in a controlled manner. The spout 24 is at the in-use upper end of the night bag 20, unlike prior art night bags which reduces contamination risk. A closable flip cap 26 has a tube portion 27, which is sealingly fitted into the spout 24. The cap element 28 is used to close the flip cap 26 via a push-fit connection. The length of the tube 21 is chosen to suit the use conditions, but is typically 1 m-1.5 m in length, and is of typical width 8 mm. At the second end 23 of the tube 21 is a connection flange 29 to enable the connection to the catheter valve or leg bag.

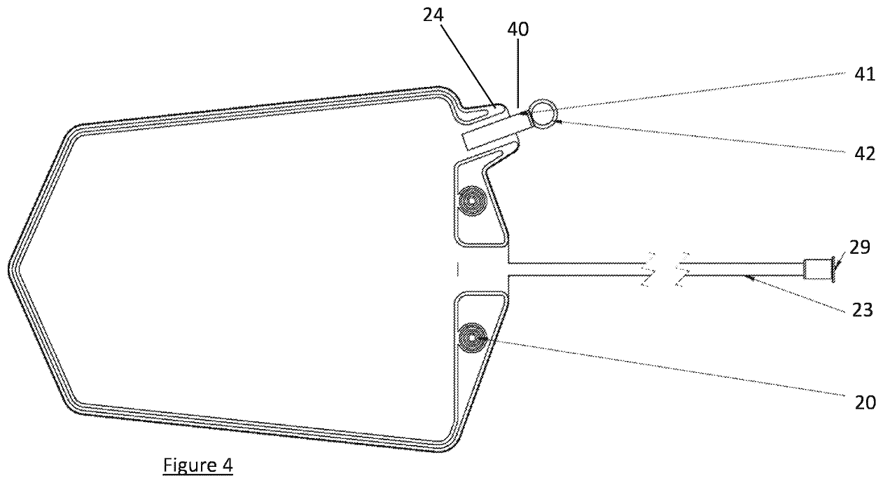

FIG. 4 illustrates the night bag 20 of FIG. 2, but with a different closure means for the spout 24. A bag stopper 40 comprises a tube portion 41 which is sealingly fitted into the spout 24. In the illustrated embodiment, the tube portion 41 has an outer diameter of 10 mm and is of length 40 mm. The in-use outer end of the bag stopper 40 is closed to prevent unwanted leakage from the night bag 20. A ring 42 is provided to assist a user in removing the bag stopper 40 when required.

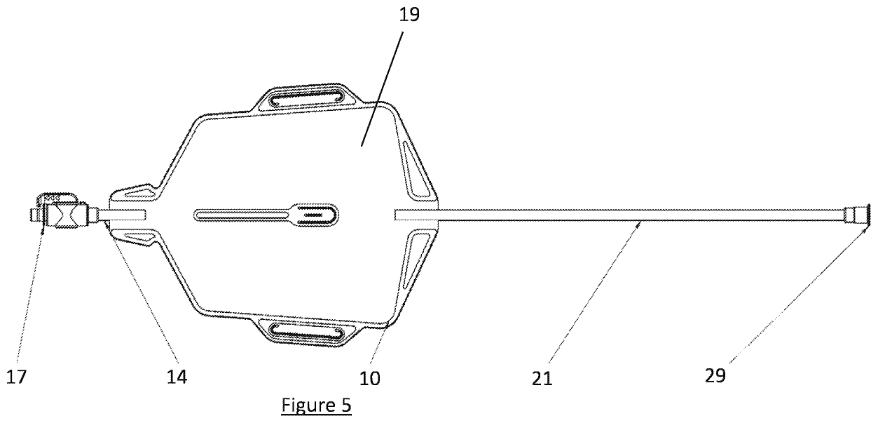

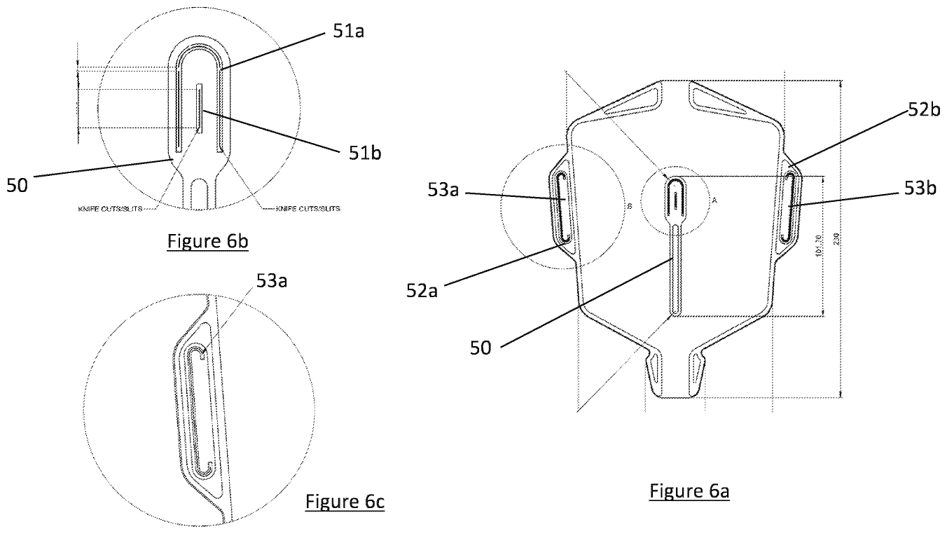

Further features of the leg bag 10 of FIG. 1 are shown in FIGS. 5 and 6. In these Figures, features shown in FIG. 1 are assigned the same numbers as in FIG. 1. The leg bag 10 has a central region 50 in which the two faces of the bag are sealed together, such that this region does not form part of the fluid collecting portion of the bag 10. Within the central region 50 is a plurality of knife cuts or slits 51a-c. The slits 51a-c pass through the region 50 and allow a tap, such as the tap 17 to be pushed through the slits 51. Once done, the surrounding material from which the region 50 is formed acts to prevent the tap from exiting the slits 51 and so allows the tap to be conveniently stowed when the bag 10 is being worn. Moreover the slits 51a-c allow the bag 10 to curve more easily and so fit better about the user's leg.

A further two regions 52a, 52b are of similar form to the region 50 and similarly each includes a slit 53a, 53b. These regions 52a, 52b can firstly act as handles to enable the bag 10 to be easily held. Secondly, the slits 53a, 53b enable the regions 52a, 52b to be curved around a user's legs more easily and with a lower restoring force acting to move the regions 52a, 52b away from the leg than would otherwise be the case. This makes fitment to a person simpler and also minimises the chance of the bag's outline being seen beneath clothing.

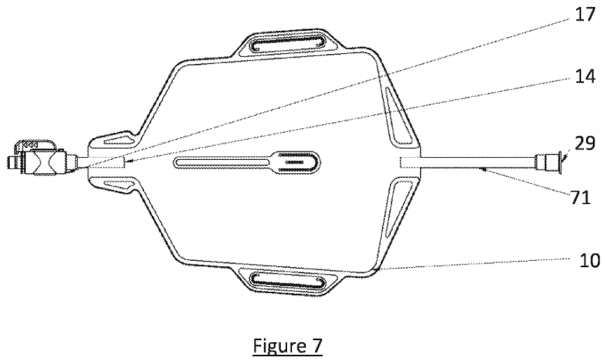

FIG. 7 illustrates a leg bag 10 in which the tube 71 connectable to the catheter, urinary or male external or condom catheter is of shorter length to enable the leg bag 10 to be worn higher up the person's leg.

FIGS. 8 and 9 illustrate a first embodiment of tap assembly 80. The tap assembly 80 is suitable for use as the tap assembly for a catheter valve. The tap assembly 80 comprises a tap body 81. The tap body 81 comprises first and second cylinders 82, 83 which mutually intersect at right angles to each other. The first cylinder 82 defines two mutually opposed apertures (one of which, 84, is shown) in the cylinder wall which allow fluid flow through the second cylinder 83 when the tap assembly 80 is in the open position. Secured to a first end 85 of the second cylinder 83 is a connector insert 86 comprising a plurality of nested frusto-conical sections 87, which in-use engage the inside of the catheter and retain the tap assembly 80 in fluid tight connection.

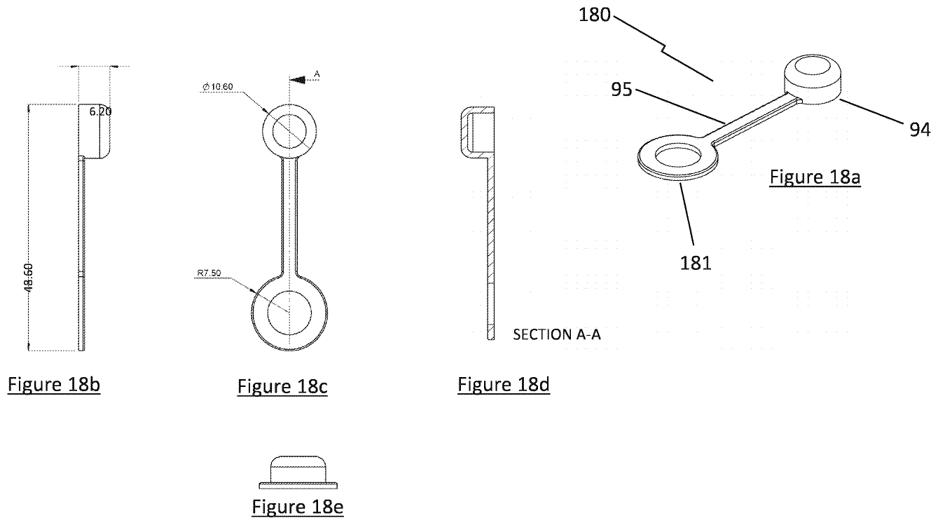

Secured to the second end 88 of the second cylinder 83 is a closure assembly which comprises a tap body tube insert 89. The first end 90 of the tap body tube insert 89 is sealingly seated in the second end 88 and has a flange ring 91 which extends over the end of the second cylinder 83. A smaller diameter cylinder 92 of the tap body tube insert 89 extends away from the second cylinder 83, the outer surface of the cylinder 92 comprising an annular recess 92a in which is seated an O-ring 93. A cap element 180 (see FIGS. 18) has a cap 94, housed at the end of a link 95 is provided to cover over the end of the cylinder 92 to ensure fluid does not leak through the tap assembly 80.

Flow of fluid through the tap assembly 80 is controlled by the tap lever 96 which is mounted to a cylindrical element 97 housed within the first cylinder 82. The cylindrical element 97 defines in its walls, two aperture (one of which, 98, is shown). When the apertures 84, 98 are aligned, then fluid can flow through the assembly 80 and out of the bag to which the tap assembly 80 is fixed. Movement of the tap lever 96 moves the apertures into and out of alignment, thus regulating the fluid flow. The tap lever 96 allows for easy operation of the tap assembly 80 for persons who have difficulty carrying out manipulative tasks. The buttons x and y prevent the tap lever 96 from turning more than 180 degrees.

One of the surfaces 105 of the free end 106 of the tap lever 96 curves in a concave direction. In the illustrated embodiment this is achieved by the tap lever 96 thickening toward the free end 106. In an alternative embodiment, not illustrated, the curvature is achieved in another manner known in the art. The curvature enables the free end 106 of the tap lever 96 to engage with the flange 29, on the opposite side of the flange 29 to which the tap assembly is connected, and resist the separation of the flange 29 from a tap assembly. The free end 106 in the embodiments shown has an optional notch 107 to better engage the flange 29.

FIGS. 10 and 11 illustrate a second embodiment of tap assembly 100,. The features of the second embodiment are as for the first embodiment. The connector insert 86 of the first embodiment is replaced however with an alternative configuration of connector insert 101 in which there is only a single frusto-conical section 102 over the end of a cylindrical element 103, which in-use engage the inside of the second tube 14 and retain the tap assembly 100 in fluid tight connection with the second tube 14.

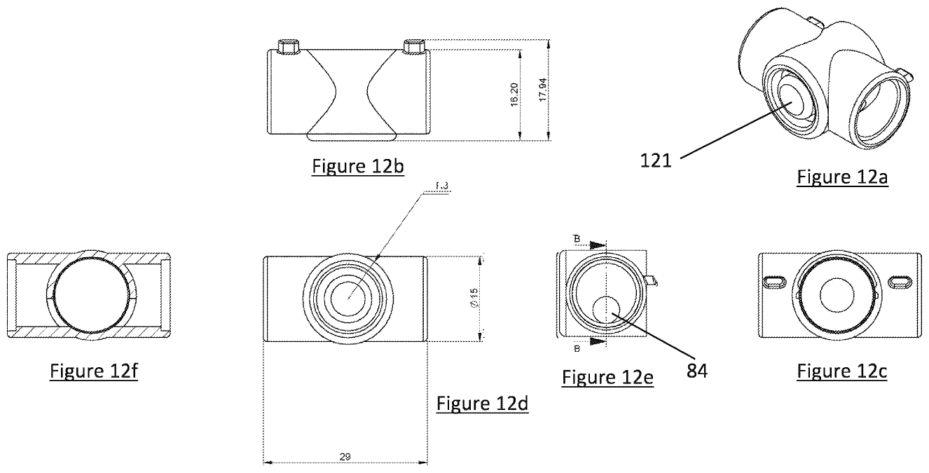

FIGS. 12a-f illustrate in more detail a tap body 81. These show the off-axis location of the apertures 84 in the wall of the first cylinder 82. Further illustrated is a receiving member 82 which is secured to the cylindrical element 97 by a push-fit joint; 121 is the housing for the cap 94 when not in use.

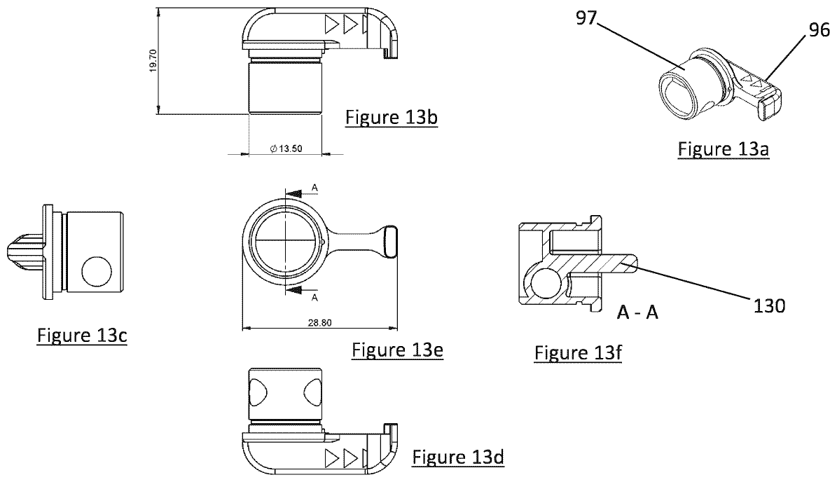

FIGS. 13a-f illustrate the tap lever 96 of FIGS. 10 and 11. The tap lever 96 is housed on a finger 130 so that pressure on the tap lever 96 causes equivalent rotational movement of the cylindrical element 97 and allows good control over the orientation of the apertures 84.

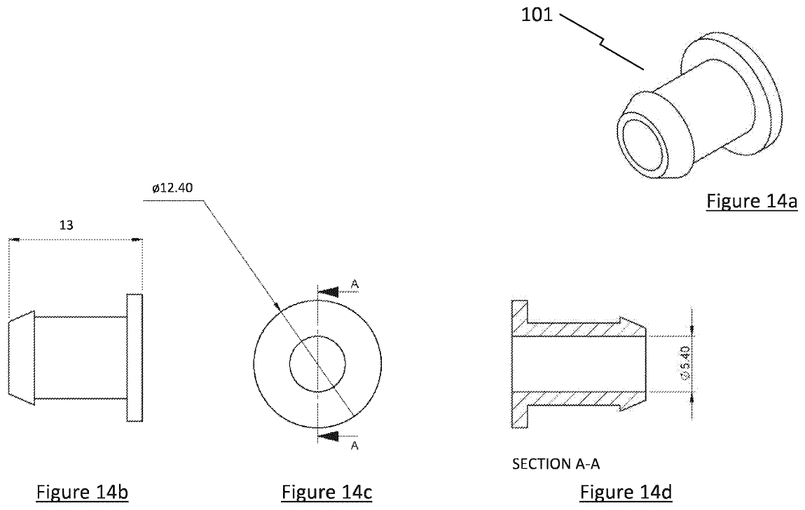

FIGS. 14a-e illustrate in more detail the insert 101 of FIGS. 10 and 11.

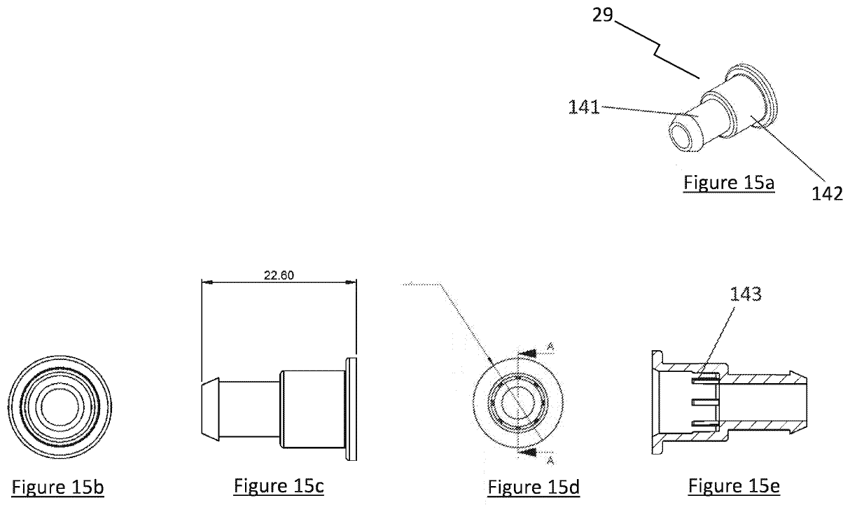

FIGS. 15a-e illustrates the connection flange 29 of FIG. 3. The connector flange 29 is similar in shape to the connector insert 101 of FIGS. 10 and 11, but includes an additional cylindrical element 141, co-axial with a first cylindrical element 142, which prevents the tube from being fitted too high and also creates a seal with the tube. Within the body of the element 142 is a plurality of ridges 143.

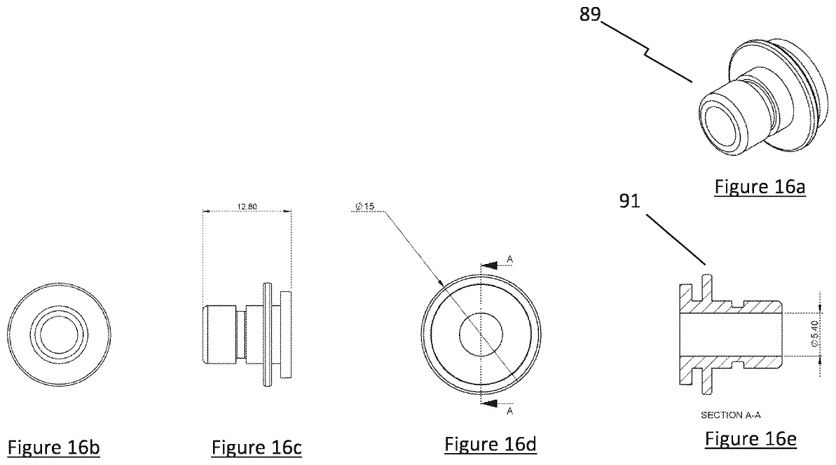

FIGS. 16a-e illustrate in more detail the tap body tube insert 89 of FIGS. 8 and 9. The tap body tube insert 89 is held in position in a similar manner to the connector insert below, with actuation of the catch by operation of the button x (see FIG. 9).

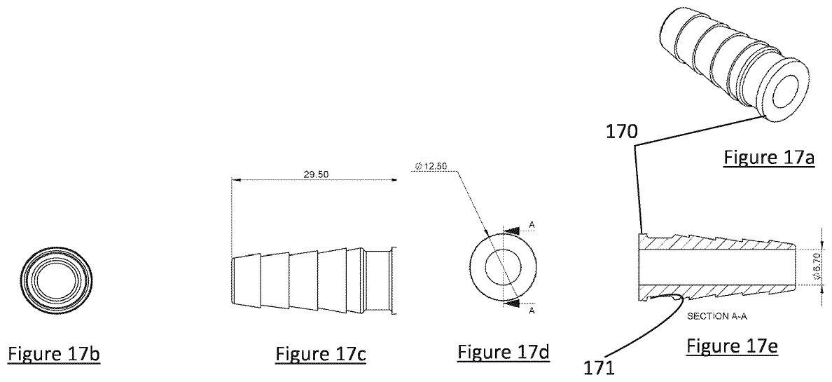

FIGS. 17a-e illustrate the connector insert 86 of FIGS. 8 and 9. The end flange 170 of the connector insert 86 is utilised to hold the connector insert 86 in position within the tap body 81. Once the connector insert 86 is in position within the tap body 81 an internal catch is actuated to extend into the second cylinder 83 and engage the rear surface 171 of the end flange 170 to hold the connector insert 86 within the second cylinder 83.

FIGS. 18a-e illustrate a cap element 180 which is utilised to close off a tap assembly to prevent fluid from exiting. The cap element has a cap 94, housed on a first end of an elongate link 95. The link 95 is formed of a flexible material which allows the cap 94 to be brought into position when required to fit over the end of the cylinder 92 of FIG. 9. The second end of the link 95 houses an annulus 181 which in-use fits around the cylinder 92 in a friction fit arrangement.

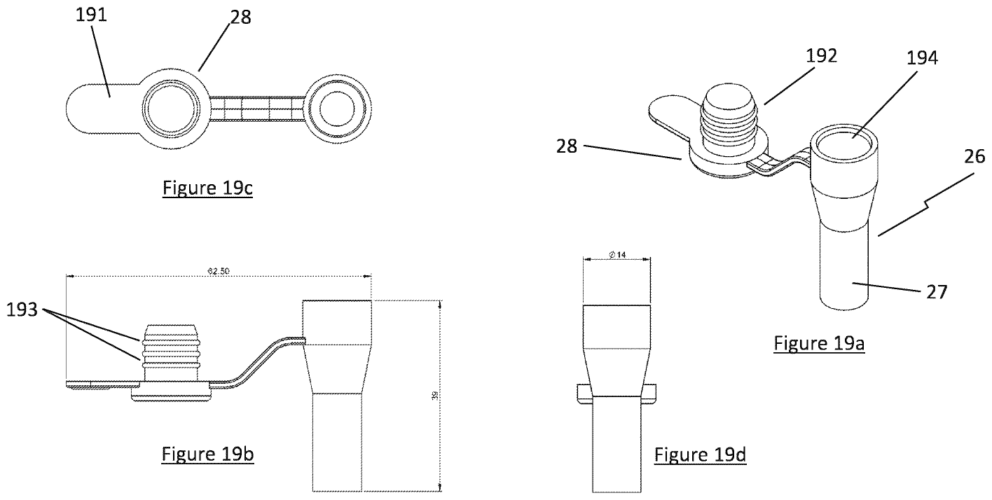

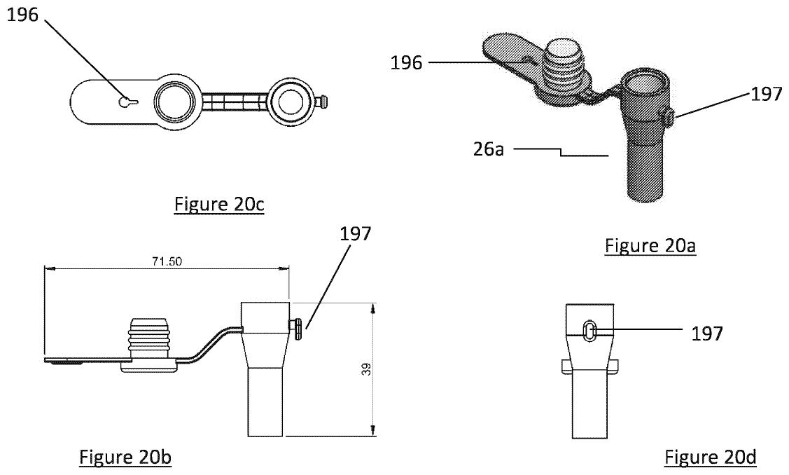

FIGS. 19a-d illustrate the bag flip cap 26 of FIG. 3 in more detail. The cap element 28 has a tab 191 which facilitates a user in positioning the flip cap 26. Further, the cap element 28 includes a plug 192, generally cylindrical in shape and having a series of concentric rings 193, axially deployed about the plug 192. The plug in-use is inserted into the open end 194 of the flip cap 26. The concentric rings 193 are to a limited extent of a resilient nature and aid in retaining the plug 192 in position and in sealing the open end 194. In an optional embodiment, shown in FIGS. 20a-d, the tab 191 includes an aperture 196 to engage a nodule 197 on the cap 26a to provide an indication that the cap 26a is in the correct position to fluidly seal and also to resist removal of the cap 26 as from the closed position.

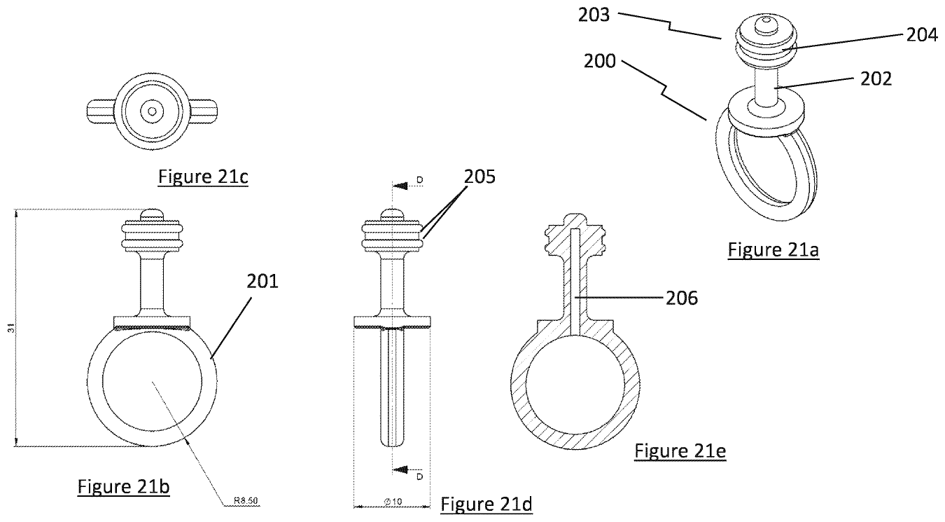

FIGS. 21a-e illustrate a further embodiment of a bag stopper 200. The bag stopper 200 is of more lightweight construction than other bag plugs, which aids in their handling. The bag stopper 200 comprises a grip ring 201 which a user utilises to hold the bag stopper 200 on removal from a catheter bag. Extending from the ring 201 is a cylinder shaft 202 of narrow diameter. The distal end 203 of the cylinder shaft 202 houses a seal element 204 comprising 2 sealing rings 205 which engage the spout of a catheter bag in a sealing engagement as the stopper 200 is inserted. The joint between the cylinder shaft 202 and the grip ring 201 is strengthened by the annulus 205, which also acts to provide an indication to a user that the stopper 200 is properly inserted as it engages the end of the spout and moreover can provide an additional fluid seal with the end of the spout. The cylinder shaft 202 is hollow along its axis 206 to allow for the plug to be machine insertable such that once it has been removed it cannot be reinserted by hand.

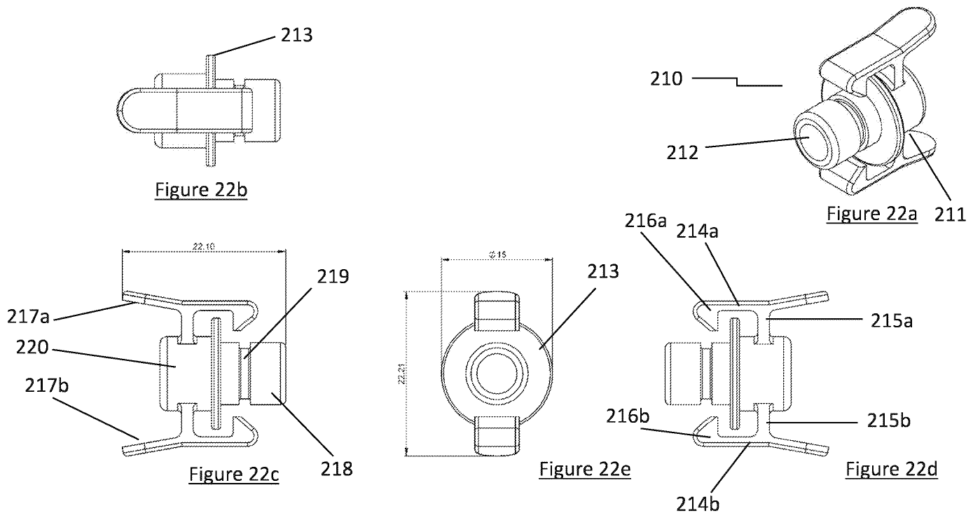

FIGS. 22a-e illustrate a connection clip 210 for use in securing tube elements, primarily an extension tube, together in a fluid-tight manner. The connection clip 210 as shown in this embodiment can be utilised where a person has limited grip, such as when wearing protective gloves utilised by care operatives dealing with the catheter bag. The central body 211 of the connection clip 210 defines a central axial lumen 212, through which fluid can flow. The connection clip 210 includes a flange 213 which supports, within the lumen 212, a sealing element to engage the end of a tube inserted into the second end 220 of the central body 211.

Securing clips 214a, 214b are each resiliently mounted on mounts 215a, 215b and biased such that the securing arms 216a, 216b are urged towards each other as shown in the FIGS. 21.

Handles 217a, 217b are provided on the securing clips 214a, 214b. When pressure is applied to each of the handles 217a, 217b the securing arms 216a, 216b are urged apart allowing a tube to pass between the securing arms 216a, 216b and about the outside of the first end 218 of the central body 211 in sealing engagement. A seal such as an O-ring can be provided in the annular groove 219 to increase the seal. Release of pressure on the handles 217a, 217b results in the securing arms being urged together and exert pressure on the tube, pushing the tube against the first end 218 and improving the seal therebetween. The securing arms 216a, 216b also act to secure a connection flange 29 between the securing arms 216a, 216b and the flange 213 so that the tube cannot accidentally be pulled off.

FIG. 23 illustrates the use of the connection clip 210 in accordance with FIGS. 22 on a length of tubing.

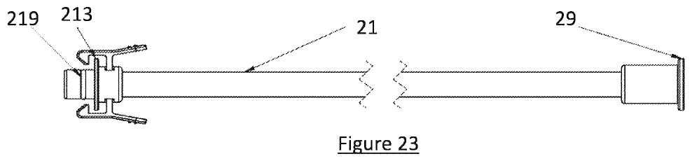

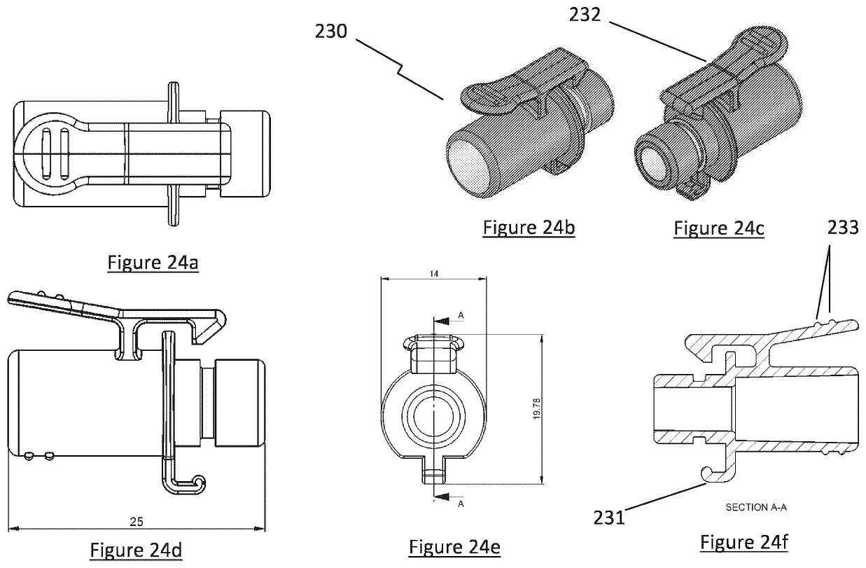

In the embodiment of connection clip, generally referenced 230 shown in FIGS. 24 the connection clip 230 has many features in common with the connection clip 210 of FIGS. 22. One of the securing clips 214 of the connection clip 210 is however replaced by a resilient arm 231 which acts to secure the flange 29 in position. The outer surface of the securing clip 232 has two ridges 233 to aid a user in applying the connection clip 230.

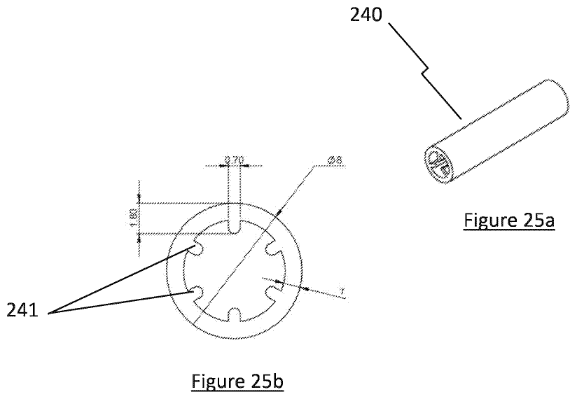

FIGS. 25a, 25b illustrate an embodiment of a non-blocking tube for use as part of the invention. As indicated above, it is important that the fluid pathways remain open to prevent fluid from being retained in the bladder or from not reaching the urine drainage bag. Because of the movement of the user, a tube can be easily bend such that flow is prevented. Moreover, when lying or sitting the weight of a user can act to close the tube if care is not taken. The tube 240 as illustrated resists such bending in that axially aligned ridges 241 are provided, circumferentially spaced about the inside surface of the tube 240. The number of ridges 241 and their height is chosen to suit the use, but should be sufficient to resist closure of the tube 240 but not such that the tube cannot easily bend. In the illustrated embodiment, six equally spaced ridges 241 are provided in a tube having an 8 mm outer diameter. Each ridge is of width 0.7 mm and height 0.8 mm.

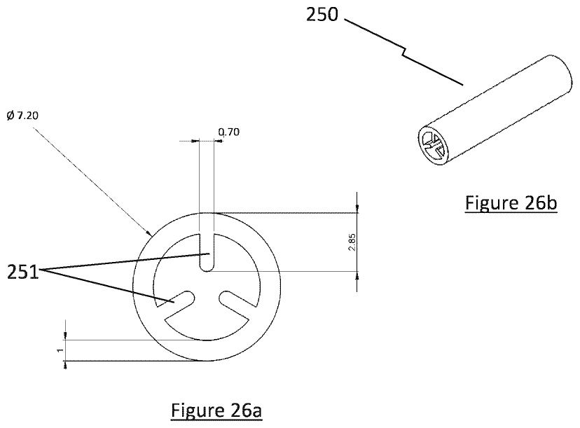

FIGS. 26a, 26b illustrate a further embodiment of non-blocking tube in which the tube 250 has 3 ridges 251 to prevent the tube from blocking. The ridges 251 extend further into the lumen of the tube 250.

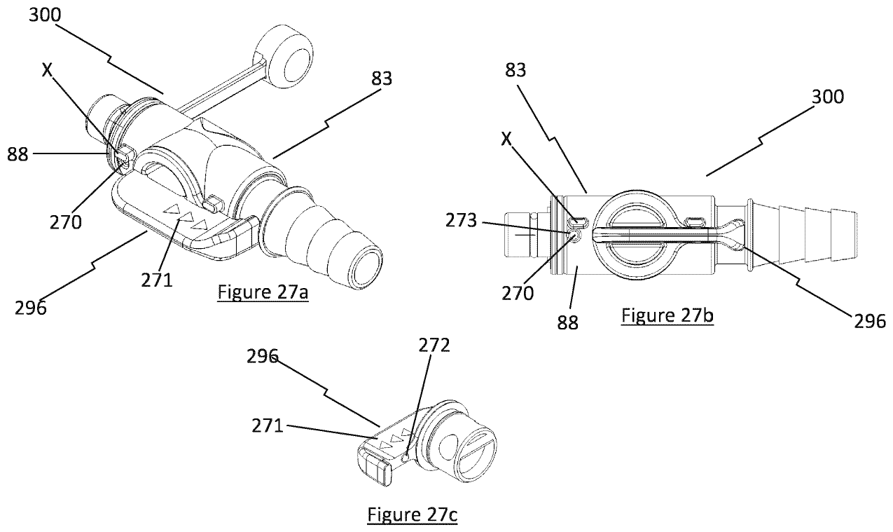

FIGS. 27a-b illustrate a third embodiment of tap assembly 300. Where a feature of the third embodiment is as for the first embodiment, then the same reference number is used. The second cylinder 83, further comprises a hemispherical recess 270. The recess 270 further comprises a rim 273 around the circumference of the recess 270. The recess 270 is situated towards the second end 88 of the second cylinder 83, situated below the button X. FIGS. 27a-c illustrate a second embodiment of the tap lever 296. Again, where a feature of the second embodiment is as for the first embodiment, the same reference number is used. The tap lever 296 has a wider main body 271 compared to the first embodiment, which in-use makes the operation of the tap lever 296 easier for those who struggle with dexterous tasks. The tap lever 296 comprises a hemispherical protrusion 272 situated on the underside of the tap lever 296 which protrusion 272 complements the shape of the hemispherical recess 270 on the second cylinder 83. The protrusion 272 protrudes from the tap lever 296 in a way that when the tap lever 296 is maneuvered into the locked alignment, the protrusion 272 slots over the rim 273 into the recess 270 with an audible click. The click acts as an audible and physical aid to ensure that the tap assembly is in the fully closed position. It will be recognised that the protrusion and the recess can shapes known in the art, other than hemispherical.

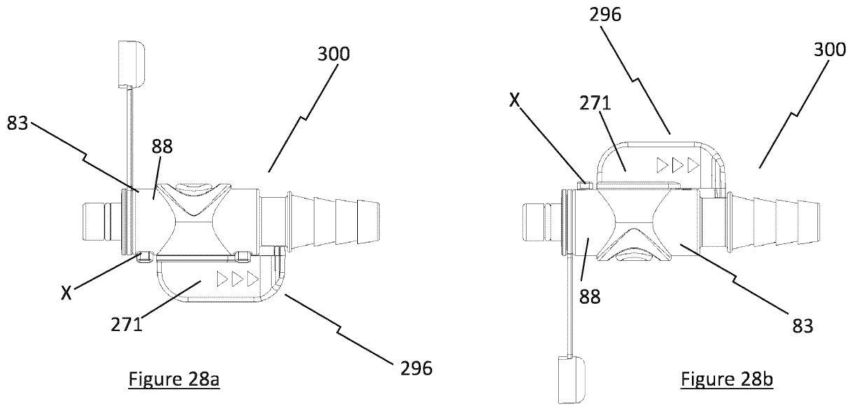

FIGS. 28a and 28b illustrate in more detail the wider main body 271 of the tap lever 296 described above in FIGS. 27a-c.

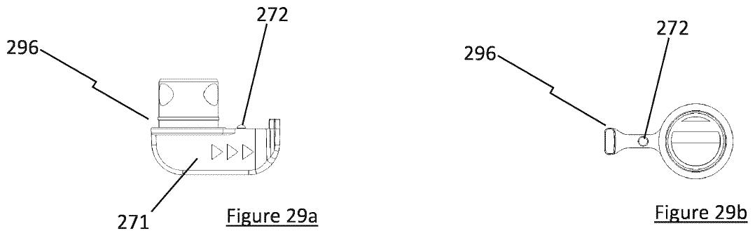

FIGS. 29a and 29b illustrate in more detail the hemispherical protrusion 272 described above in FIGS. 27a-c.

1. A tap assembly for a urine drainage system, the tap assembly comprising:
a) a tap body formed as a first and a second cylindrical bodies, mutually intersecting at right-angles, the wall of the first cylindrical body defining mutually opposed apertures, aligned with the axis of the second cylindrical body;
b) a tap member rotatably housed in the first cylinder movable between a first position preventing fluid flow along the second cylindrical body and a second position allowing fluid flow, the tap member actuable by a tap lever and
c) the tap lever comprising a protrusion, the protrusion engaging, in use, a recess on the second cylindrical body, the recess being of complementary shape to the protrusion.

2. A tap assembly according to claim 1, wherein the tap lever has a curved surface.

3. A tap assembly according to claim 1, wherein the tap lever comprises a notch.

4. A tap assembly according to claim 1, wherein the recess includes a rim around the circumference of the recess.

5. A tap assembly according to claim 1, wherein a first connector extends from a first end of the second cylindrical body.

6. A tap assembly according to claim 1, wherein the first connector comprises a plurality of nested frusto-conical sections.

7. A tap assembly according to claim 6, wherein the first connector has a single frusto-conical section.

8. A tap assembly according to claim 1, wherein a re-closable closure extends from a second end of the second cylindrical body.

9. A urine drainage system comprising a tap assembly according to claim 1.
